(19) United States
(12) Patent Application Publication
(10) Pub. No.: US 2003/0125239 A1
(43) Pub. Date: Jul. 3, 2003

(54) Compositions for drug delivery

(76) Inventors: Andrea Crisanti, London (GB); Selma Esseghir, London (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/262,209

(22) Filed: Sep. 30, 2002

Related U.S. Application Data
(63) Continuation-in-part of application No. PCT/GB01/01699, filed on Oct. 18, 2001.

(30) Foreign Application Priority Data
Feb. 2, 2001 (GB) 0102667.3
Aug. 7, 2002 (GB) 0218324.2

(51) Int. Cl. A61K 48/00; C12N 9/22

(52) U.S. Cl. 514/7; 514/8; 435/199; 530/358

(57) ABSTRACT

A serum-free composition comprises a conjugate of a DNA-binding protein, or a fragment thereof, and a polynucleotide. The composition is suitable for intramuscular administration, to treat disease.

### FIELD OF THE INVENTION

The present invention relates to the preparation of proteins as transfection agents, particularly, but not exclusively, in the form of histone H1 protein/nucleic acid complexes.

### BACKGROUND TO THE INVENTION

Gene therapy provides the potential to cure selected genetic diseases. However, a major obstacle is the effective delivery of the gene or protein of interest to the target site. A variety of viral and non-viral vectors have been developed to deliver genes or gene products to various cells, tissues and organs by ex vivo or in vivo strategies. Among viral-based vectors, retroviruses, adenoviruses, adeno-associated viruses and herpes viruses have been most extensively studied. Among non-viral-based vectors, liposomes and cationic lipid-mediated systems have been used to introduce plasmic DNA directly into animals. However, one of the main challenges of gene therapy remains the design of effective delivery systems.

Histones have also been proposed for use as a vehicle for gene delivery. Histones are the proteins responsible for the nucleosomal organisation of chromosomes in eukaryotes. The core histones H2A, H2B, H3 and H4 form the core structure of the nucleosome, and the linker histone H1 seals two rounds of DNA at the nucleosomal core.

Zaitsev et al., Gene Therapy (1997)4, 586-592 discloses certain nuclear proteins, including histone, which can be prepared to act as DNA carriers for gene transfer. The example disclosed is calf-thymus histone H1 which is prepared in a serum-containing media with calcium ions required to obtain high transfection efficiencies. Chloroquine is also present to obtain efficient transfection. However, the presence of serum and chloroquine makes this formulation unsuitable for clinical applications.

Haberland et al., Biochimica et Biophysica Acta, 1999; 1445: 21-30, discloses that histones require Ca²⁺ to achieve high transfection efficiency. In the absence of Ca²⁺, chloroquine was required.

EP-A-0908521 discloses a transfection system for the transfer of nucleic acids into cells. Transfection is achieved using histones which bind to polynucleotides and then transfer the DNA into the cell.

Fritz et al., Human Gene Therapy, 1996; 7: 1395-1404, also uses DNA-binding histone to transfect DNA. However, this system also requires lipofectin to enhance transfection efficiency. Lipofectin is toxic and is generally unsuitable for therapeutic applications.

Schwartz et al., Gene Therapy, 1999; 6:282-292, discloses a transfection system based on cationic lipids. The system requires the DNA to be transported to be first compacted using histone peptides. The compacted DNA/histone complex is then brought into contact with the cationic lipid and used in the transfection process.

WO-A-89/10134 discloses chimeric peptides for neuropeptide delivery through the blood-brain barrier. The chimeric peptides comprise a neuropeptide and a peptide capable of crossing the blood-brain barrier via receptor-mediated transcytosis. Histone is mentioned as a peptide that fulfills this criteria. The chimeric peptide is produced via chemical linkage, so that on crossing the blood-brain barrier, the linkage is broken to release the neuropeptide.

### SUMMARY OF THE INVENTION

The present invention is based on the surprising finding that histone proteins and other DNA-binding proteins can be prepared and used to transfect in serum-free conditions. The present invention is also based on the identification of a histone H1 peptide that is an efficient transfection agent.

According to one aspect of the present invention, a composition comprises a conjugate of a DNA-binding protein, or a fragment thereof, and a polynucleotide, wherein the composition is substantially free of serum, calcium ions and chloroquine.

Surprisingly, it has been found that DNA-binding proteins, for example histone H1, can act as efficient transfection agents when prepared and used in serum-free media. The conjugates can be administered to a patient in a suitable composition without requiring the presence of calcium ions, which can induce a painful reaction on administration, or chloroquine, which is toxic. The use of lipofectin (cationic lipids) is also not required.

According to a second aspect of the invention, a DNA-binding protein or a peptide as defined above, is used in the manufacture of a therapeutic composition for intramuscular or intra-dermal administration, for the delivery of a polynucleotide across a cellular membrane, the composition being free of serum, calcium ions and chloroquine.

According to a third aspect of the invention, a histone protein for the transfection of a polynucleotide has less than 200 amino acids and comprises the amino acid sequence identified herein as SEQ ID NO. 2. This peptide may be used in the presence or absence of other components, e.g. calcium.

### DESCRIPTION OF THE INVENTION

The present invention provides compositions comprising delivery vehicles with ability to transport polynucleotides across a cell membrane to effect entry of the polynucleotides into the cell or across an intracellular compartment.

In the context of the present invention, the term “transfection” refers to the delivery of a polynucleotide, e.g. DNA, to inside a cell.

In one aspect of the present invention the conjugates are comprised in a composition lacking serum, calcium ions, chloroquine and cationic lipids. Although the mechanism is unknown, the presence of serum in the composition significantly reduces the effectiveness of transfection.

The composition is intended preferably for administration via intramuscular or intra-dermal delivery. This is because the muscle tissue comprises little natural serum constituents which may otherwise interfere with the transfection efficiency. Administration by the intramuscular route may be achieved using techniques known to those skilled in the art. Injection directly into the muscle tissue is a suitable delivery method, as is needle-less injection methods.

Although the compositions are free of serum, calcium ions, cationic lipids and chloroquine, other suitable diluents or excipients may be present. Suitable buffers, excipients and diluents will be apparent to the skilled person. If the therapeutic agent to be delivered is an immunogen (or encodes an immunogen) the composition may also comprise an adjuvant, e.g. alum, that helps promote an immunogenic response. Suitable adjuvants will be apparent to the skilled person.

DNA-binding proteins which may be used in the present invention will be apparent to the skilled person.

The DNA-binding proteins must be capable of permitting transfection. This can be tested simply by the techniques known in the art, and disclosed herein. In a preferred embodiment, the protein is a histone protein. Preferably the histone is the linker histone H1. H1 histones exist in many different isoforms, although high levels of sequence homology exists. Preferably the histone is a human histone as this is less immunogenic. The amino acid sequence of a suitable human H1 histone is identified in Albig et al., Genomics, 1991; 10(4): 940-948. The sequences are also available on the NCBI database (Genebank Accession number M60748).

The histone may be in a truncated form, preferably in a form identified below. Having the histone in the truncated form identified below allows recombinant forms to be produced to a high level by expression in a bacterial or mammalian (or other) cell. It also allows synthetic methods to be used which avoids the need for time-consuming purification steps. Truncated forms may also be less immunogenic.

Other suitable proteins that may be used in the invention include those identified as cationic proteins in Zaitsev supra, e.g. HMG1, HMG2 and HMG17. Again, truncated forms of these proteins that retain the ability to transfect are within the scope of the present invention.

Functional variants of the proteins may also be used. For example, proteins with high levels (greater than 70%, preferably greater than 90%) of sequence similarity or identity are within the scope of the present invention. The variants may be produced using standard recombinant DNA techniques such as site-directed mutagenesis. The variants may also have conserved amino acid substitutions, e.g. replacement of a hydrophobic residue for a different hydrophobic residue. All this will be apparent to the skilled person, based on conventional protein technology. The variants must retain the functional ability to initiate transfection of a polynucleotide across a cellular membrane.

In a preferred embodiment of the invention, there is a recombinant human histone H1 peptide of less than 200 amino acid residues and comprising or consisting of the amino acid sequence identified as SEQ ID NO. 2. The peptide preferably consists of the amino acid sequence shown in SEQ ID NO. 2. This histone peptide can be used as an efficient transfection agent in the absence or presence of other additional components, including calcium ions. The peptide may be used in its monomeric form, although dimer, trimer, etc, forms are also envisaged.

Variations on this sequence are also envisaged, and these are referred to herein as “functional homologues”. A functional homologue is a protein/peptide that has at least 80% sequence identity, preferably at least 90% identity, across its length to at least a part of the sequence identified herein as SEQ ID NO. 2, and which retains the ability to transfect polynucleotides. Sequence identity may be determined using the BlastX programme available from NCBI, using the default settings.

The polynucleotide to be transported may comprise any suitable nucleic acid, e.g. DNA or RNA, in either single-stranded or double-stranded form..

The polynucleotide acid may encode a therapeutic agent, e.g. an enzyme, toxin, immunogen, etc. or may itself be the therapeutic agent. For example, anti-sense RNA may be used to target and disrupt expression of a gene. All this will be apparent to the skilled person.

In a preferred embodiment, the polynucleotide is RNAi. RNAi molecules are double-stranded RNA molecules, used for targeted gene suppression in cells. Typically, RNAi molecules are produced synthetically and are approximately 15-30, more usually approximately 20 base pairs in length (Tuschl, Nature Biotech, 2002; 20: 446-448). The preparation of suitable RNAi molecules is described in Paul et al, Nature Biotech., 2002; 29: 505-508, and in Miyagishi et al., Nature Biotech., 2002; 19: 497-500. Using the RNAi molecules it is possible to knock out the activity of specific genes. This facilitates the identification, validation and characterisation of new drugs and their mode of action.

The polynucleotide may also be in the form of a vector or plasmid. As used herein, vector (or plasmid) refers to discrete elements that are used to introduce heterologous DNA into cells for either expression or replication thereof. Selection and use of such vehicles are well known to the skilled person. Many vectors are available, and selection of appropriate vector will depend on the intended use of the vector, e.g. whether it is to be used for DNA amplification or for DNA expression, the size of the DNA to be inserted into the vector, and the host cell to be transformed with the vector. Each vector contains various components depending on its function (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

Additional cell transportation signals may be present on the DNA-binding protein. For example, nuclear localisation signals may be an additional component of the constructs. This may aid the transport of the therapeutic component to the correct intracellular location.

The preparation of suitable conjugates may be carried out using conventional methods. A suitable DNA-binding protein or peptide, e.g. Histone, may be prepared using known protein purification methods. The purified protein may then be bound with the DNA. The ratio of protein to DNA may be optimised by the skilled person, and may vary depending on the DNA, treatment etc.

It is apparent that the compositions of the invention are intended for therapeutic use. Therapy includes prophylactic treatments, e.g. vaccination.

Applications for the compositions of the present invention include:

1. Gene therapy.

Gene therapy may include any one or more of: the addition, the replacement, the deletion, the supplementation, the manipulation etc. of one or more nucleotide sequences in, for example, one or more targeted sites—such as targeted cells. If the targeted sites are targeted cells, then the cells may be part of a tissue or an organ. General teachings on gene therapy may be found in Molecular Biology, Ed Robert Meyers, Pub VCH, such as pages 556-558.

By way of further example, gene therapy can also provide a means by which any one or more of: a nucleotide sequence, such as a gene, can be applied to replace or supplement a defective gene; a pathogenic nucleotide sequence, such as a gene, or expression product thereof can be eliminated; a nucleotide sequence, such as a gene, or expression product thereof, can be added or introduced in order, for example, to create a more favourable phenotype; a nucleotide sequence, such as a gene, or expression product thereof can be added or introduced, for example, for selection purposes (i.e. to select transformed cells and the like over non-transformed cells); cells can be manipulated at the molecular level to treat, cure or prevent disease conditions such as cancer (Schmidt-Wolf and Schmidt-Wolf, 1994, Annals of Hematology 69; 273-279) or other disease conditions, such as immune, cardiovascular, neurological, inflammatory or infectious disorders; antigens can be manipulated and/or introduced to elicit an immune response, such as genetic vaccination. In a particularly preferred embodiment, the compositions may be used to introduce functional proteins in the cytoplasm of genetically deficient cell types.

2. Cancer therapy.

The compositions may be used to transport into cancer cells polynucleotides that are or encode transcription factors, and which are able to restore cell cycle control or induce differentiation. For example, it is understood that many cancer cells would undergo apoptosis if a functional P-53 molecule is introduced into their cytoplasm. The present invention may be used to deliver polynucleotides that encode such gene products.

3. Use in expression systems.

For example, it is desirable to express exogenous proteins in eukaryotic cells so that they get processed correctly. However, many exogenous proteins are toxic to eukaryotic cells. In manufacturing exogenous proteins it is therefore desirable to achieve temporal expression of the exogenous protein. The system may therefore be used in connection with an inducible promoter for this or any other application involving such a system.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may further comprise any suitable binder(s), lubricant(s), suspending agent(s), coating agent(s), solubilising agent(s), and other carrier agents that may aid or increase entry into the target site.

The delivery of one or more therapeutic genes according to the invention may be carried out alone or in combination with other treatments or components of the treatment. Diseases which may be treated include, but are not limited to: cancer, neurological diseases, inherited diseases, heart disease, stroke, arthritis, viral infections and diseases of the immune system. Suitable therapeutic genes include those coding for tumour-suppressor proteins, enzymes, pro-drug activating enzymes, immunomodulatory molecules, antibodies, engineered immunoglobulin-like molecules, conjugates, hormones, membrane proteins, vasoactive proteins or peptides, cytokines, chemokines, anti-viral proteins, antisense RNA and ribozymes.

The amount to be administered to a patient will depend on the usual factors: age of the patient, weight, severity of the condition, route of administration, activity of the therapeutic etc. All this can be determined by conventional methods known to the skilled person.

The following Examples illustrate the invention.

### EXAMPLE 1

The histone protein used in this Example is a histone fragment designated herein as histone H1.4, and was prepared from the human linker Histone H1 gene (GeneBank Accession Number M60748; and the protein used is identified herein as SEQ ID NO. 1). The gene was expressed in bacteria and the protein was purified under denaturing conditions and then refolded in phosphate buffer at acidic pH.

Transfection experiments were carried out by mixing increasing amounts (μg) of the partial linker Histone 1.4 protein with 2 μg of the reporter plasmid pGL 3-c (Promega), which encodes the luciferase gene. Different weight to weight ratios of protein-DNA complex were prepared in Tris-saline pH 8. HeLa cells were washed with media and incubated in either:

(1) 1 ml of media with 10% serum;

(2) 1 ml of media with 10% serum and 2 mM calcium;

(3) 1 ml of media with 10% serum and 100 μM chloroquine; or

(4) 1 ml of media without any serum.

The protein/DNA complexes were incubated in the appropriate cell media overnight.

The cells were lysed and luciferase enzyme activity measured by the Promega luciferase assay kit using a luminometer. The results showed that transfection in media without serum was relatively high, reaching the order of 10⁶ relative light units (RLU). Transfections in media with the serum produced very low values of luciferase expression, but increased transfection was observed when the media was supplemented with chloroquine or calcium. The results are shown in Table 1.

TABLE 1

| | | RLU (48 hours post- |
|---|---|---|
| | Conditions | transfection) |
| Background | | 43 |
| HeLa cells | | 67 |
| DNA (2 μg) | | 669 |
| Lipofectin (5 μl) | without FCS | |
| (2 μg DNA) | | |
| PGL3-c | | 8 287 184 |
| 0.2/1 Histone/pGL3-c | | 4 622 214 |
| pGL3-c | 2 mM Ca²⁺ | 72* high cell death |
| pGL3-c | 100 μM Chloroquine | 60 448 012 |
| Histone/pGL3-c ratio | without FCS | |
| (2 μg DNA) | | |
| 0.4/1 | | 57 370 |
| 0.8/1 | | 9 368 937 |
| 1.6/1 | | 103 515 |
| | with FCS | |
| 0.2/1 | | 600 |
| 0.4/1 | | 395 |
| 0.8/1 | | 266 |
| 1.2/1 | | 5516 |
| 1.6/1 | | 1219 |
| | in 2 mM Ca²⁺ with FCS | |
| 0.2/1 | | 440 337 |
| 0.4/1 | | 61 264 344 |
| 0.8/1 | | 25 733 564 |
| 1.2/1 | | 15 155 237 |
| 1.6/1 | | 12 791 919 |
| | in 100 μM Chloroquine | |
| | with FCS | |
| 0.2/1 | | 871 |
| 0.4/1 | | 1 340 |
| 0.8/1 | | 1 920 860 |
| 1.2/1 | | 298 493 |
| 1.6/1 | | 392 291 |

The peptide fragment (SEQ ID NO. 2) was also tested, but this failed to transfect in the absence of calcium ions. However, in the presence of calcium, this human recombinant peptide showed very good transfection efficiency, as shown in the following Example.

### EXAMPLE 2

Gambiae Sua 4.0 cells and HeLa cells were grown in Schneiders Drosophila medium and DMEM (GIBCO) respectively. Both mediums were supplemented with 10% foetal calf serum (FCS), 100U ml⁻¹ penicillin and 100 μg ml⁻¹ streptomycin. Cells were grown at 25° C. (Gambiae Sua 4.0), 37° C. and 10% CO₂(HeLa) and passaged every two to three days to maintain exponential growth. The day before transfection 5×10⁴ HeLa cells/well or 3×10⁵ Sua 4.0 cells/well were seeded on a 24-well plate.

Transfection experiments were performed using histone H1.4F (SEQ ID NO. 2) and Lipofectin Reagent (GibcoBRL, Life Technologies), as a control.

For transfections, H1.4F protein (SEQ ID NO. 2) was diluted in a solution of 135 mM NaCl, 20 mM Tris-HCl pH 8 to obtain a final protein concentration of 0.05 μg/μl. For each transfection point, 0.5 μg - 4 μg of vector-based RNAi or in-vitro synthesized RNAi (RNAi was synthesized in-vitro using the AmpliScribe™ kit (EPICENTRE) according to the manufacturer's instructions and specifications) and target plasmid encoded DNA were complexed with diluted H1.4F protein by mixing them in a sterile polysterene tube at 1:1, 1:2, 1:3, 1:4, 1:5, and 1:6 polynucleotide-protein ratios (w/w). H1.4F protein was added drop wise while mixing. CaCl₂ was added to the mixture at a final concentration of 2 mM. The mixture was allowed to stand at room temperature for 30 min.

Cells were washed once with complete medium and then 800 to 900 μl of corresponding medium was added to each well. Each medium was supplemented with FCS and CaCl₂ (optimal final concentrations should be determined for each individual experiment; recommended range 2-10 mM).

The RNAi-(or RNAi-vector-based)-DNA-protein complexes were placed onto cells and the cells incubated for 6 hours or overnight (about 14-16 hours) at 37° C. and 10% CO₂ for HeLa cells or 25° C. for Gambiae Sua 4.0. At this stage a precipitate may cover the cells, however this will not affect or impede transfection.

At the end of the incubation time the polynucleotide-protein-containing medium was replaced with 1 ml of normal growth medium containing serum and the cells were incubated for a further 24-48 hrs.

The cells were analysed for reduction of EGFP expression by fluorescence microscopy. In order to enhance detection of transfected cells, the media was replaced with 1 ml of phosphate buffered saline (PBS). Cells are examined at wavelength 490 nm to detect EGFP expression and 580 nm to detect RFP expression, when appropriate.

The results are shown in Table 2.

TABLE 2

| Conditions | Duplicate in relative light units (RLU) | | AVG |
|---|---|---|---|
| negative control | 244 | | 244 |
| naked DNA | 74 | | 74 |
| lipofectin | 618093 | 377657 | 497875 |
| ratio 1-1, 4 mM | 2242258 | 3718512 | 2980385 |
| ratio 1-1, 6 mM | 56802 | 66410 | 61606 |
| ratio 1,1 8 mM | 6795 | 10513 | 8654 |
| ratio 1-2, 4 mM | 562067 | 1479912 | 1020990 |
| ratio 1-2, 6 mM | 52734 | 66658 | 59696 |
| ratio 1-2, 8 mM | 16128 | 78614 | 47371 |
| ratio 1-3, 4 mM | 455511 | 778229 | 616870 |
| ratio 1-3, 6 mM | 27188 | 50745 | 38966.5 |
| ratio 1-3, 8 mM | 12077 | 30425 | 21251 |

The results show that the histone peptide facilitated greater transfection of polynucleotide at the optimal concentration than the lipofectin control.

We claim:

1. A composition comprising a conjugate of a polynucleotide and a histone protein, or a fragment of the protein which retains the ability to bind to a polynucleotide and undergo transfection, the composition being free of serum, calcium ions, chloroquine and cationic lipids.

2. The composition, according to claim 1, wherein the histone protein is histone H1.

3. A histone protein for the transfection of a polynucleotide, having less than 200 amino acid residues and comprising the amino acid sequence defined herein as SEQ ID NO. 2, or a functional homologue thereof.

4. The histone protein, according to claim 3, consisting of the amino acid sequence defined herein as SEQ ID NO. 2.

5. A composition comprising a histone protein having less than 200 amino acid residues and comprising the amino acid sequence defined herein as SEQ ID NO. 2, or a functional homologue thereof; wherein said composition further comprises a polynucleotide.

6. The composition, according to claim 5, wherein the polynucleotide is DNA.

7. The composition, according to claim 5, wherein the polynucleotide is RNA.

8. The composition, according to claim 7, wherein the polynucleotide is RNAi.

9. The composition, according to claim 5, wherein the histone protein consists of SEQ ID NO. 2.

10. A method for treating disease wherein said method comprises administering, to a patient in need of such treatment, an effective amount of a composition comprising a conjugate of a polynucleotide and a histone protein, or a fragment of the protein which retains the ability to bind to a polynucleotide and undergo transfection, the composition being free of serum, calcium ions, chloroquine and cationic lipids.

11. The method, according to claim 10, wherein administration is intramuscular or intra-dermal.

12. The method, according to claim 10, wherein the histone protein is histone H1.

13. A method for providing therapy wherein said method comprises administering, to a patient in need of therapy, a composition comprising a histone protein, having less than 200 amino acid residues and comprising the amino acid sequence defined herein as SEQ ID NO. 2, or a functional homologue thereof; wherein said composition further comprises a polynucleotide.

14. The method, according to claim 13, wherein the histone protein consists of the amino acid sequence defined herein as SEQ ID NO. 2.

15. The method, according to claim 13, wherein the polynucleotide is DNA.

16. The method, according to claim 13, wherein the polynucleotide is RNA.

17. The method, according to claim 16, wherein the polynucleotide is RNAi.


SEQUENCE LISTING (2 sequences)

SEQ ID NO: 1 - protein, 234 aa, Homo sapiens
MSETAPAAPAAPAPAEKTPVKKKARKSAGAAKRKASGPPVSELITKAVAASKERSGVSLAALKKALAAAGYDVEKNNSRIKLGLKSLVSKGTLVQTKGTGASGSFKLNKKAASGEAKPKAKKAGAAKAKKPAGAAKKPKKATGAATPKKSAKKTPKKAKKPAAAAGAKKAKSPKKAKAAKPKKAPKSPAKAKAVKPKAAKPKTAKPKAAKPKKAAAKKKKLEQKLISEEDLKLN

SEQ ID NO: 2 - protein, 130 aa, Homo sapiens
LVQTKGTGASGSFKLNKKAASGEAKPKAKKAGAAKAAKKPAGAAKKPKKATGAATPKKSAKKTPKKAKKPAAAAGAKKAKSPKKAKAAKPKKAPKSPAKAKAVKPKAAKPKTAKPKAAKPKKAAAKKKKL